(19) United States
(12) Patent Application Publication
(10) Pub. No.: US 2025/0050047 A1
(43) Pub. Date: Feb. 13, 2025

(54) GAS FLOW CONTROL DEVICE

(71) Applicants: The United States Government as represented by the Department of Veterans Affairs, Washington, DC (US); The Board of Trustees of the University of Illinois, Urbana, IL (US)

(72) Inventors: Israel Rubinstein, Chicago, IL (US); Andrea Benedetti, Chicago, IL (US); Alexander Twaddle, Chicago, IL (US); Grace Dyer, Chicaco, IL (US); Kimberlee Wilkens, Palos Park, IL (US); Lara Nammari, Urbana, IL (US)

(21) Appl. No.: 18/719,658

(22) PCT Filed: Dec. 12, 2022
(86) PCT No.: PCT/US2022/052526

§ 371 Date: Jun. 13, 2024

Related U.S. Application Data
(60) Provisional application No. 63/289,053, filed on Dec. 13, 2021.

(51) Int. Cl. A61M 16/06; A61M 16/00

(52) U.S. Cl. CPC A61M 16/0666; A61M 16/0003; A61M 16/0051; A61M 16/022; A61M 16/0683; A61M 2202/0007; A61M 2202/02; A61M 2205/0227; A61M 2205/0233; A61M 2205/103; A61M 2205/18; A61M 2205/3327; A61M 2205/3331; A61M 2205/3561; A61M 2205/3592; A61M 2205/50; A61M 2205/8206; A61M 2209/088; A61M 2210/0618

(57) ABSTRACT

A gas flow control device uses the curvature of nasal cannula or other tubes conveying the gas to control an actuator movable between two different configurations to permit or prevent gas flow. Curvature of the nasal cannula is measured by a sensor whose electrical resistance changes in response to the curvature of the sensor. The sensor is mounted on the cannula. Signals from the sensor are transmitted to a controller, which moves the actuator when the curvature exceeds or falls below a threshold value as measured by the change in electrical resistance of the sensor. The device finds use in oxygen therapy, where oxygen flow to a patient is cut off when the sensor indicates non-compliant behavior by the patient, indicating release of oxygen into the environment and causing a potential fire hazard.

### CROSS REFERENCE TO RELATED APPLICATIONS

This application is based upon and claims benefit of priority to U.S. Provisional Application No. 63/289,053, filed Dec. 13, 2021, which application is hereby incorporated by reference herein.

### FIELD OF THE INVENTION

The invention concerns a device for controlling the flow of gas such as oxygen from a gas source to a patient through gas delivery devices such as nasal cannula, nasal pillows and masks.

### BACKGROUND

Oxygen therapy involves the prescribed administration of oxygen and/or a flow rate for oxygen to be delivered for effective breathing via a nasal cannula having prongs resting in the nose, via a face mask, or via a tube placed in the trachea of a patient.

Home oxygen therapy has been shown to decrease mortality and improve quality of life for patients with Chronic Obstructive Pulmonary Diseases (COPD) and hypoxemia. Home oxygen therapy is prescribed to those who have been lifelong smokers, and some who are actively smoking. For home use, oxygen concentrators are used to pull oxygen from the surrounding air, never requiring a refill, but its use is dictated by a prescribed flow rate for each patient.

For portable use, oxygen is considered a medicine stored as a gas or liquid in special tanks. Lightweight concentrators are also available, eliminating the need for a special tank.

Because oxygen accelerates combustion and is a known fire hazard, COPD patients who smoke and use oxygen therapy in their homes or other enclosed spaces risk having a fire-related incident.

The prongs of a cannula are intended to direct oxygen into the nose. However, a significant amount of oxygen exits the nose and constantly leaks out and bathes the lower face. An oxygen-enriched environment facilitates ignition and combustion of any material. The buildup of oxygen in an enclosed space is often caused by non-compliant behavior, such as removing one's cannula without turning off the machine. Additionally, patients suffering from hypoxemia due to, for example, congestive heart failure and COPD are at risk when nasal cannula are not used properly. For example, a relatively large proportion of COPD patients using continuous supplemental oxygen suffer nasal cannula dislodgment during sleep on a weekly basis. Nasal cannula dislodgement predisposes COPD patients to exacerbations that may require emergency room treatment. There is clearly an opportunity to increase the safety of oxygen therapy by reducing the potential for fire-related incidents as well as correcting dislodgement of nasal cannula and thereby mitigating concomitant complications.

### SUMMARY

The invention concerns a device for controlling flow of gas from a gas source to a patient. In an example embodiment the device comprises a nasal cannula providing fluid communication between the gas source and the patient. The nasal cannula includes two prongs adapted to fit within nostrils of the patient. A first and a second portion of the nasal cannula are in fluid communication with the prongs. The first and second portions are adapted to fit respectively between a first and a second ear and head of the patient. A first sensor is positioned on the first portion of the nasal cannula. The first sensor is adapted to sense a degree of curvature of the first portion of the nasal cannula and generate first signals indicative thereof. An actuator is engaged with the nasal cannula between the gas source and the first and second portions of the nasal cannula. The actuator has a first configuration permitting flow of gas from the gas source through the nasal cannula to the first and second portions of the nasal cannula, and a second configuration preventing flow of gas from the gas source through the nasal cannula to the first and second portions of the nasal cannula. A controller is in communication with the first sensor and the actuator. The controller is adapted to receive the first signals from the first sensor and adjust the actuator between the first and second configurations. The controller adjusts the actuator to the first configuration when the first sensor senses the degree of curvature greater than a threshold value, and the controller adjusts the actuator to the second configuration when the first sensor senses the degree of curvature less than the threshold value.

The invention further encompasses a device for controlling flow of gas from a gas source to a patient. An example device comprises a tube providing fluid communication between the gas source and the patient. An adapter is in fluid communication with the tube. The adapter is engagable with a mouth and/or nose of the patient. A band extends from the adapter and is positionable surrounding a head of the patient for attaching the adapter thereto. A first sensor is positioned on the band. The first sensor is adapted to sense a degree of curvature of the band and generate first signals indicative thereof. An actuator is engaged with the tube between the gas source and the adapter. The actuator has a first configuration permitting flow of gas from the gas source through the tube to the adapter, and a second configuration preventing flow of gas from the gas source through the tube to the adapter. A controller is in communication with the first sensor and the actuator. The controller is adapted to receive the first signals from the first sensor and adjust the actuator between the first and second configurations. The controller adjusts the actuator to the first configuration when the first sensor senses the degree of curvature greater than a threshold value. The controller adjusts the actuator to the second configuration when the first sensor senses the degree of curvature less than the threshold value.

By way of example, the controller may be adapted to generate an alarm signal when the first sensor senses the degree of curvature less than the threshold value.

In an example embodiment, the first sensor may comprise a resistive flexible sensor having an electrical resistance which changes in response to a change in curvature of the first sensor. For example, the electrical resistance may increase with an increase in curvature of the first sensor.

An example embodiment may further comprise a second sensor positioned on the second portion of the nasal cannula. The second sensor is adapted to sense a degree of curvature of the second portion of the nasal cannula and generate second signals indicative thereof. The controller is in communication with the second sensor. The controller is adapted to receive both the first and second signals respectively from the first and second sensors and adjust the actuator between the first and second configurations. The controller adjusts the actuator to the first configuration when at least one of the first and second sensors sense the degree of curvature greater than a threshold value. The controller adjusts the actuator to the second configuration when both the first and second sensors sense the degree of curvature less than the threshold value.

In an example embodiment the second sensor may comprise a resistive flexible sensor having an electrical resistance which changes in response to a change in curvature of the second sensor. By way of example, the electrical resistance of the second sensor may increase with an increase in curvature of the second sensor.

In an example embodiment the actuator comprises a servo motor having a rotatable shaft and a head mounted on the shaft. The head has at least two fingers extending therefrom. The fingers are in spaced apart relation and define a slot therebetween. The nasal cannula or the tube are received within the slot. The head is rotatable between the first configuration wherein the nasal cannula or the tube extend through the slot without deformation thereby permitting flow of the gas therethrough, and the second configuration, wherein the fingers impinge on the nasal cannula or the tube so as to form a kink therein thereby preventing flow of the gas therethrough.

By way of example the controller may comprise a microprocessor. A wireless communication system may be used to provide communication between the controller and the first sensor and the actuator. Also by way of example, a first electrical conductor may connect the controller to the first sensor and a second electrical conductor may connect the controller to the actuator.

An example device according to the invention may further comprise a power supply electrically connected to the first sensor, the actuator and the controller for providing electrical power thereto. By way of example the power supply may comprise a battery.

In an example embodiment the adapter comprises a mask or nasal pillows.

The invention also includes a method of controlling flow of a gas in response to non-compliant behavior of a patient with respect to a nasal cannula. In an example embodiment the method comprises:
measuring a curvature of a first portion of the nasal cannula;
determining if the curvature is greater than or less than a threshold value;
halting flow of the gas if the curvature of the first portion of the nasal cannula is less than the threshold value;
allowing flow of the gas if the curvature of the first portion of the nasal cannula is greater than the threshold value.

An example method may further comprise:
measuring a curvature of a second portion of the nasal cannula;
determining if the curvature of the second portion of the nasal cannula is greater than or less than a threshold value;
halting flow of the gas if the curvatures of both the first and second portions of the nasal cannula are less than the threshold value;
allowing flow of the gas if one of the curvatures of the first and second portions of the nasal cannula is greater than the threshold value.

Another example method of controlling flow of a gas in response to non-compliant behavior of a patient with respect to an adapter in fluid communication with a source of the gas, the adapter being engagable with a mouth and/or nose of the patient, a band extending from the adapter and positionable surrounding a head of the patient for attaching the adapter thereto, comprises:
measuring a curvature of the band;
determining if the curvature is greater than or less than a threshold value;
halting flow of the gas if the curvature of the band is less than the threshold value;
allowing flow of the gas if the curvature of the band is greater than the threshold value.

In an example embodiment the curvature is measured by an electrical resistance of a sensor. The electrical resistance is proportional to a curvature of the sensor. In an example embodiment the electrical resistance increases with an increase of the curvature of the sensor.

### BRIEF DESCRIPTION OF THE DRAWINGS

FIG. 1 is an isometric illustration of a patient using an example device for controlling gas flow according to the invention;

FIG. 2 is a schematic diagram showing a device for controlling gas flow being used by a patient with examples of different gas sources;

FIGS. 3 and 4 are schematic diagrams depicting compliant and non-compliant behavior of a patient when using an example device according to the invention;

FIG. 5 is an isometric view of an example curvature sensor used with devices according to the invention;

FIG. 6 is an isometric view of an example actuator used with devices according to the invention;

FIG. 7 is an isometric view of an example component used with the actuator shown in FIG. 6;

FIGS. 8 and 9 are plan views which show the actuator respectively in a first configuration permitting gas flow and a second configuration preventing gas flow;

FIG. 10 is an isometric view of an example controller used with devices according to the invention; and

FIGS. 11-14 are isometric views of patients using another embodiment of a device for controlling fluid flow according to the invention.

### DETAILED DESCRIPTION

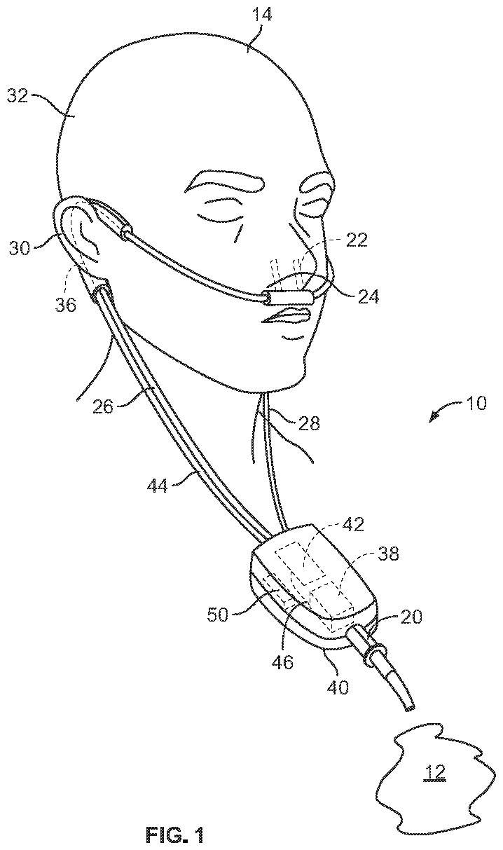

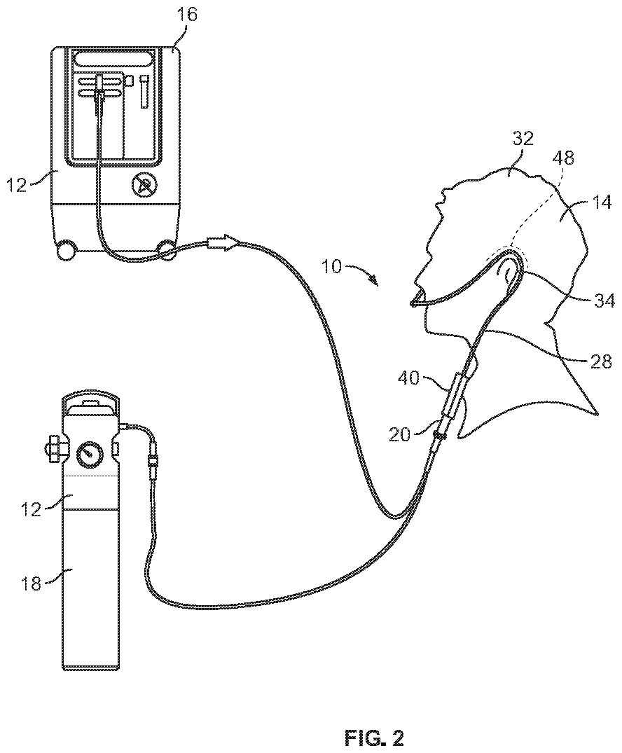

FIGS. 1 and 2 show an example device 10 according to the invention for controlling flow of gas from a gas source 12 to a patient 14. As shown in FIG. 2, potential gas sources include gas concentrators 16 and cylinders of compressed or liquid gas 18. While examples in this specification emphasize oxygen as the gas, it is understood that other medical gases as well as air may also be used with the device 10. Furthermore, device 10 is compatible with low, medium and high gas flow systems.

The example device 10 is shown in FIG. 1 in detail and comprises a nasal cannula 20 providing fluid communication between the gas source 12 and the patient 14. The nasal cannula 20 includes two prongs 22 adapted to fit within nostrils 24 of the patient 14. A first portion 26 and a second portion 28 of the nasal cannula 20 are in fluid communication with the prongs 22. The first portion 26 is adapted to fit between a first ear 30 and the head 32 of the patient 14 and the second portion 28 is adapted to fit between a second ear 34 and the head 32 of the patient 14 as shown in FIG. 2.

A first sensor 36 is positioned on the first portion 26 of the nasal cannula 20 also between the first ear 30 and the head 32. The first sensor 36 is adapted to sense a degree of curvature of the first portion 26 of the nasal cannula 20 and generate first signals indicative thereof. An actuator 38, positioned within a housing 40 through which the nasal cannula 20 passes, engages the nasal cannula 20 between the gas source 12 and the first and second portions 26 and 28 of the nasal cannula 20. As described in detail below, the actuator 38 has a first configuration permitting flow of gas from the gas source 12 through the nasal cannula 20 to the first and second portions 26, 28 of the nasal cannula 20, and a second configuration preventing flow of gas from the gas source 12 through the nasal cannula 20 to the first and second portions 26, 28 of the nasal cannula 20. A controller 42, also contained within housing 40, is in communication with the first sensor 36 and the actuator 38. Communication between the controller 42, the actuator 38 and the first sensor 36 may be via a wireless system, such as Bluetooth technology, or hard wired and comprising a first electrical conductor 44 connecting the controller 42 to the first sensor 36 and a second electrical conductor 46 connecting the controller 42 to the actuator 38 as shown in FIG. 1.

Controller 42 is adapted to receive the first signals from the first sensor 36 indicative of the curvature of the first portion 26 of the nasal cannula 20 and adjust the actuator 38 between the first and second configurations. In this example embodiment, the controller 42 adjusts the actuator 38 to the first configuration (permitting gas flow) when the first sensor 36 senses a degree of curvature of the first portion 26 of the nasal cannula 20 greater than a threshold value. Curvature above the threshold value indicates that the patient 14 is wearing the nasal cannula 20 properly, as evidenced by the first portion 26 of the nasal cannula 20 curving around the first ear 30. Oxygen or other medical gases are thus being properly consumed. The controller 42 adjusts the actuator 38 to the second configuration (preventing gas flow) when the first sensor 36 senses a degree of curvature of the first portion 26 of the nasal cannula 20 less than the threshold value. Curvature less than the threshold value indicates a straightening of the first portion 26 of the nasal cannula 20, a result of the prongs 22 no longer being within the nostrils 24, as would occur, for example if the patient 14 removed the prongs and placed them on the forehead (see FIG. 4). If oxygen were to continue to flow in this situation a dangerous condition would arise, as an oxygen rich environment may form, especially in an enclosed space, risking a fire-related incident.

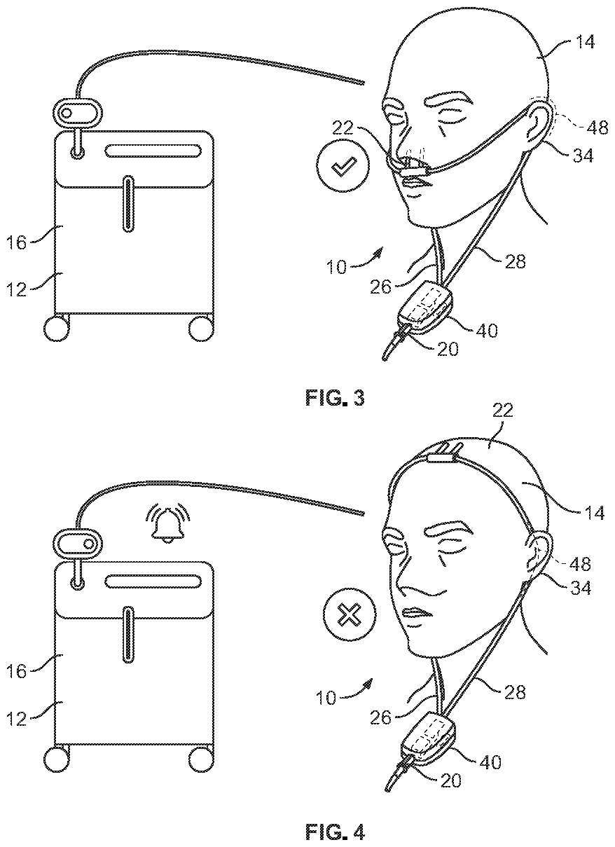

As shown in FIGS. 3 and 4, the controller 42 may also be adapted to generate an alarm signal when the first sensor 36 senses the degree of curvature less than the threshold value, indicative of non-compliant behavior. FIG. 3 shows the patient 14 using device 10 in conjunction with a gas concentrator 16. As device 10 is being properly worn in FIG. 3, actuator 38 is in the first configuration permitting oxygen flow from the source 12 and no alarm is signaled. However, as shown in FIG. 4, patient 14 has placed the prongs 22 on the forehead, allowing oxygen to flow freely into the environment. First sensor 36 detects the change in curvature of the first portion 26 of the nasal cannula 20 resulting from the new position of the prongs 22 and signals from the first sensor 36 to the controller 42 indicative of the non-compliant behavior result in the controller moving the actuator 38 to the second configuration to halt oxygen flow. Contemporaneously, controller 42 generates an alarm signal which is wirelessly transmitted and registered at the gas concentrator 16, audibly and/or visually, so that remedial action might be taken.

It is thought advantageous for device 10 to comprise a second sensor 48 positioned on the second portion 28 of the nasal cannula 20 worn behind the second ear 34 as shown in FIGS. 2, 3 and 4. The second sensor 48 is adapted to sense a degree of curvature of the second portion 28 of the nasal cannula 20 and generate second signals indicative thereof. Controller 42 is in communication with the second sensor 48 (wirelessly or hardwired) and is adapted to receive both the first and second signals respectively from the first and second sensors 36 and 48 and adjust the actuator 38 between the first and second configurations. In this example embodiment having two sensors 36 and 48, the controller is programmed to adjust the actuator 38 to the first configuration (permitting gas flow) when at least one of the first and second sensors 36 and 48 sense a degree of curvature greater than a threshold value, indicating compliant behavior by the patient 14. The controller 38 is further programmed to adjust the actuator to the second configuration (preventing gas flow) when both the first and second sensors 36 and 48 sense a degree of curvature less than the threshold value, indicative of non-compliant behavior by patient 14. It is expected that using two sensors 36 and 48 to measure curvature of both the first and second portions 26 and 28 of the nasal cannula 20 and predicating action by the controller 42 to command the actuator 38 into the second configuration based upon the signal from both sensors indicating non-compliant behavior will lessen the likelihood of false alarms and unnecessary gas cut-offs.

As shown in FIG. 1, device 10 further comprises a power supply 50 electrically connected to the first sensor 36 (and the second sensor 48 when present), the actuator 38 and the controller 42 for providing electrical power to these components. In this example embodiment, power supply 50 comprises an electrical battery positioned within the housing 40.

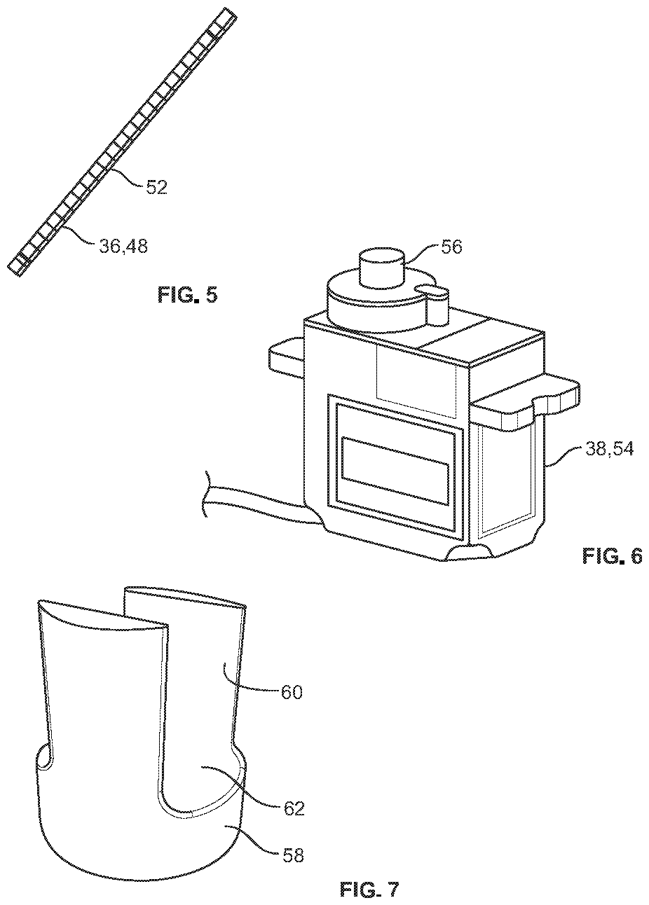

FIG. 5 shows example first and second sensors 36 and 48, which comprise an elongate, electrically resistive flexible substrate 52 having an electrical resistance which changes in response to a change in curvature of the substrate. Such sensors, known as “Flex Sensors”, are commercially available from Spectral Symbol of West Valley City, Utah. The electrical resistance of the substrate 52 in this example increases with an increase in curvature of the substrate, and the electrical resistance is coordinated with the controller 42 to identify the aforementioned threshold value of the curvature, which corresponds to a particular resistance of the substrate 52. The threshold value of curvature/resistance may furthermore be calibrated to a standard or custom calibrated to a specific patient.

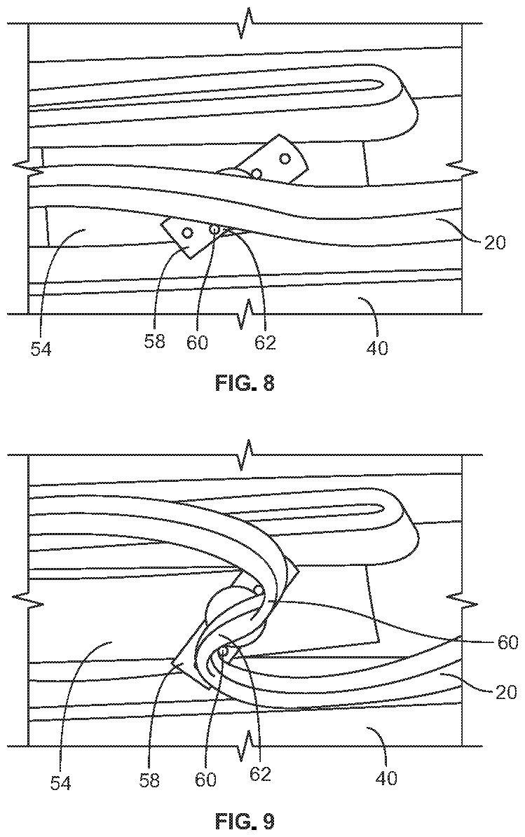

FIG. 6 shows an example actuator 38 comprising a 9g servo motor 54. Servo motor 54 has a rotatable shaft 56 on which a head 58, shown in FIG. 7, is mounted. Head 58 has at least two fingers 60 extending therefrom. Fingers 60 are in spaced apart relation and define a slot 62 between them. As shown in FIG. 8, when the servo motor 54 is positioned within the housing 40 the nasal cannula 20 or other tube is received within the slot 62. As shown in a comparison of FIGS. 8 and 9, the head 58 is rotatable between the first configuration (FIG. 8) wherein the nasal cannula 20 extends through the slot without deformation thereby permitting flow of the gas therethrough. FIG. 9 shows the head 58 rotated into the second configuration, wherein the fingers 60 impinge on the nasal cannula 20 so as to form a kink therein thereby preventing flow of the gas therethrough.

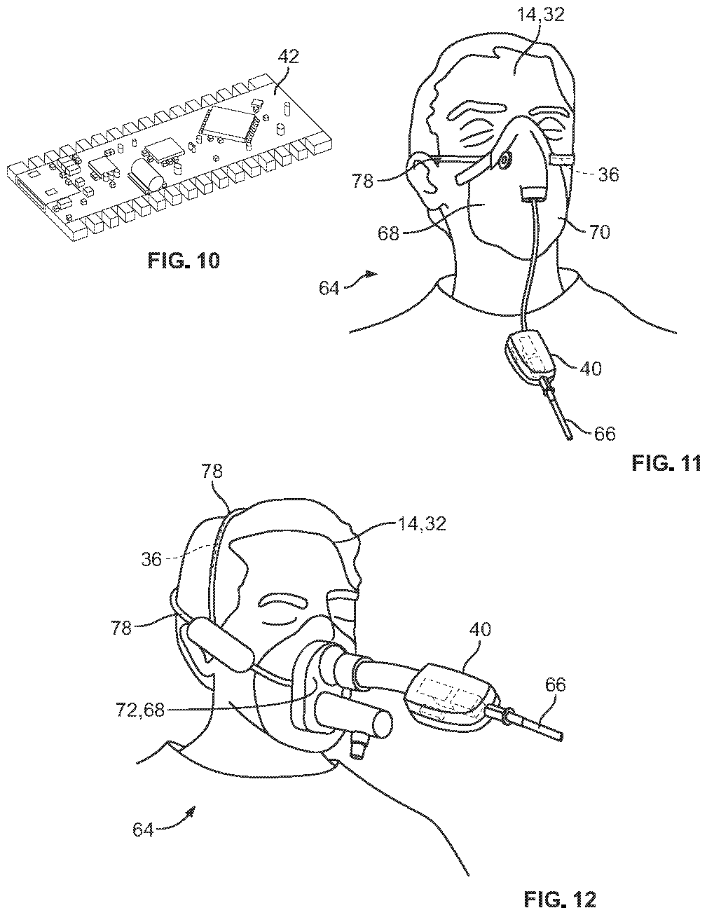

FIG. 10 shows an example controller 42, in this case a microprocessor such as a single board micro controller available from Arduino.

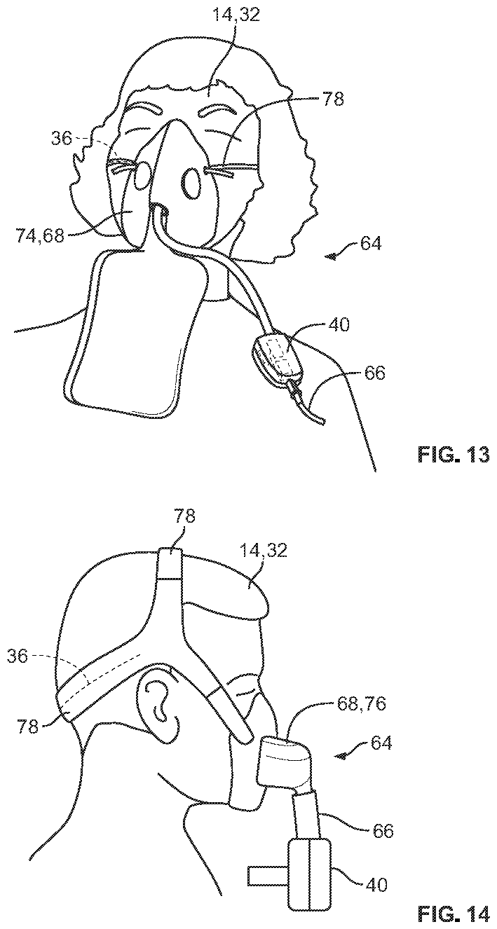

FIGS. 11-14 show another example embodiment of a device 64 for controlling gas flow according to the invention. Device 64 comprises a tube 66 providing fluid communication between a gas source (not shown) and the patient 14. An adapter 68 is in fluid communication with the tube 66. The adapter 68 is engagable with the mouth and/or nose of the patient 14 and may comprise, for example, a simple mask 70 (FIG. 11), a CPAP mask 72 (FIG. 12) a venturi mask 74 (FIG. 13) or nasal pillows 76 (FIG. 14).

One or more bands 78 extend from the adapter 68 and are positionable surrounding the head 32 of the patient 14 for attaching the adapter. A first sensor 36, as described above, is positioned on the band or bands 78. Again, the first sensor is adapted to sense a degree of curvature of the band 78 and generate first signals indicative thereof. An actuator 38 (not shown) within housing 40 as described above is engaged with the tube 66 between the gas source and the adapter 68. Again, the actuator 38 has a first configuration permitting flow of gas from the gas source through the tube 66 to the adapter 68, and a second configuration preventing flow of gas from the gas source through the tube 66 to the adapter 68 (see FIGS. 8 and 9). Controller 42 within housing 40, as described above, is in communication with the first sensor 36 and the actuator 38. The controller 42 adapted to receive the first signals from the first sensor 36 and adjust the actuator 38 between the first and second configurations. The controller 42 adjusts the actuator 38 to the first configuration when the first sensor 36 senses the degree of curvature greater than a threshold value and to the second configuration when the first sensor 36 senses the degree of curvature less than the threshold value. Curvature of the band or bands 78 is indicative of compliant or non-compliant behavior of the patient 14 as described above for the portions 26 and 28 of the cannula, and the relative straightness or curvature of the band allows for the flow of gas to the patient when the device 64 is being worn in a compliant manner (indicated by curvature above a threshold value) as well as halting gas flow when the device 64 is not worn, or not worn in a compliant manner (indicated by curvature below a threshold value).

The invention further encompasses methods for controlling gas flow in response to non-compliant behavior of a patient. In one example, drawn to a patient using a device comprising nasal cannula in fluid communication with a source of the gas, the method comprises:
measuring a curvature of a first portion of the nasal cannula;
determining if the curvature is greater than or less than a threshold value;
halting flow of the gas if the curvature of the first portion of the nasal cannula is less than the threshold value;
allowing flow of the gas if the curvature of the first portion of the nasal cannula is greater than the threshold value.

This example method may further comprise:
measuring a curvature of a second portion of the nasal cannula;
determining if the curvature of the second portion of the nasal cannual is greater than or less than a threshold value;
halting flow of the gas if the curvatures of both the first and second portions of the nasal cannula are less than the threshold value;
allowing flow of the gas if one of the curvatures of the first and second portions of the nasal cannula is greater than the threshold value.

Another example method is directed to controlling flow of a gas in response to non-compliant behavior of a patient using a device comprising an adapter (such as a mask) in fluid communication with a source of the gas. The adapter is engagable with a mouth and/or nose of the patient, and a band extends from the adapter and is positionable surrounding a head of the patient for attaching the adapter thereto. An example of such a method comprises:
measuring a curvature of the band;
determining if the curvature is greater than or less than a threshold value;
halting flow of the gas if the curvature of the band is less than the threshold value;
allowing flow of the gas if the curvature of the band is greater than the threshold value.

In either example method described above, the curvature of the nasal cannula or the band may be measured by an electrical resistance of a sensor wherein the electrical resistance is proportional to a curvature of the sensor. In an example embodiment, the electrical resistance of the sensor may increase with an increase of the curvature of the sensor. The sensor is mounted on the cannula, tube or band and thus the curvature of the sensor is the same or proportional to the curvature of the cannula tube or band.

Devices as claimed and described herein are expected to prevent injury to patients and damage to property by signaling (via an alarm) a dangerous condition and/or stopping the flow of oxygen within the device, thus preventing unintentional, excessive oxygen concentration levels that serve as an accelerant for sources of ignition. The devices according to the invention are expected to mitigate the risk and danger associated with non-compliance during oxygen therapy sessions through a self-correcting and user independent product. Since non-compliant behavior may be defined as the removal of one's cannula while the gas source is still providing oxygen, resulting in a buildup of oxygen levels in an enclosed space, devices herein are expected to achieve greater patient safety by preventing the deleterious effects of patient non-compliance.

Devices according to the invention can be added on to existing cannulas to sense the removal of the cannula. Oxygen is always flowing if systems and components herein are worn correctly, whereas non-compliant behavior triggers an existing alarm within oxygen concentrators, alerting users to either: 1) shut off the machine, or 2) put the cannula back on. In addition, devices as described and claimed herein can be paired to a select an oxygen source either directly or indirectly, such as via a smartphone, iPad, computer station, or the like.

It is advantageous if the various components of the devices described herein, as appropriate, are formed of non-toxic materials operationally safe components, and single use (disposable) components. Devices according to the invention are furthermore well adapted for use with both stationary and portable gas sources. The devices according to the invention may be used with a wide range of patients such as a neonate, infant, a child, an adolescent or an adult. Patients may be treated at home, or when confined to a medical facility or nursing home. Devices according to the invention are furthermore compact and lightweight, allowing for their use by ambulatory patients. The devices are adaptable for continuous or intermittent use.

Devices as claimed and described herein are expected to serve clinical needs for respiratory therapists, pulmonologists, nursing homes/care centers, durable medical equipment (DME) companies/staff, first responders (EMT, Firefighters, Police), insurance companies, hospitals and the like.

Additionally, the devices can be configured to selectively connect with existing FireSafe devices or other similar safety systems in a home, hospital, or nursing facility. To this end, wired or wireless signals can be generated and sent to FireSafe devices to indicate an alarm has been detected or the flow of oxygen has been obstructed.

A number of advantages are expected to be achieved by the devices described and claimed herein, including: lower oxygen (and other medical gas) use and waste; prevention of oxygen level build up without an alarm; allow for corrective measures when nasal cannula are dislodged during sleep; reduced fire risk; increased patient safety; increased safety for family, caregivers, neighbors, and pets. The devices described and claimed herein can be configured to work with existing home oxygen concentrators to trigger alarms and auto shut-off functions.

All of the embodiments of the claimed invention described herein are provided expressly by way of example only. Innumerable variations and modifications may be made to the example embodiments described herein without departing from the concept of this disclosure. Additionally, the scope of this disclosure is intended to encompass any and all modifications and combinations of all elements, features, and aspects described in the specification and claims, and shown in the drawings. Any and all such modifications and combinations are intended to be within the scope of this disclosure.

1. A device for controlling flow of gas from a gas source to a patient, said device comprising:
a nasal cannula providing fluid communication between said gas source and said patient, said nasal cannula including two prongs adapted to fit within nostrils of said patient, a first and a second portion of said nasal cannula in fluid communication with said prongs being adapted to fit respectively between a first and a second ear and head of said patient;
a first sensor positioned on said first portion of said nasal cannula, said first sensor adapted to sense a degree of curvature of said first portion of said nasal cannula and generate first signals indicative thereof;
an actuator engaged with said nasal cannula between said gas source and said first and second portions of said nasal cannula, said actuator having a first configuration permitting flow of gas from said gas source through said nasal cannula to said first and second portions of said nasal cannula, and a second configuration preventing flow of gas from said gas source through said nasal cannula to said first and second portions of said nasal cannula;
a controller in communication with said first sensor and said actuator, said controller adapted to receive said first signals from said first sensor and adjust said actuator between said first and second configurations, said controller adjusting said actuator to said first configuration when said first sensor senses said degree of curvature greater than a threshold value, said controller adjusting said actuator to said second configuration when said first sensor senses said degree of curvature less than said threshold value.

2. A device for controlling flow of gas from a gas source to a patient, said device comprising:
a tube providing fluid communication between said gas source and said patient;
an adapter in fluid communication with said tube, said adapter engagable with a mouth and/or nose of said patient;
a band extending from said adapter and positionable surrounding a head of said patient for attaching said adapter thereto;
a first sensor positioned on said band, said first sensor adapted to sense a degree of curvature of said band and generate first signals indicative thereof;
an actuator engaged with said tube between said gas source and said adapter, said actuator having a first configuration permitting flow of gas from said gas source through said tube to said adapter, and a second configuration preventing flow of gas from said gas source through said tube to said adapter;
a controller in communication with said first sensor and said actuator, said controller adapted to receive said first signals from said first sensor and adjust said actuator between said first and second configurations, said controller adjusting said actuator to said first configuration when said first sensor senses said degree of curvature greater than a threshold value, said controller adjusting said actuator to said second configuration when said first sensor senses said degree of curvature less than said threshold value.

3. The device according to claim 1, wherein said controller is adapted to generate an alarm signal when said first sensor senses said degree of curvature less than said threshold value.

4. The device according to claim 1, wherein said first sensor comprises a resistive flexible sensor having an electrical resistance which changes in response to a change in curvature of said first sensor.

5. The device according to claim 4, wherein said electrical resistance increases with an increase in curvature of said first sensor.

6. The device according to claim 1, further comprising a second sensor positioned on said second portion of said nasal cannula, said second sensor adapted to sense a degree of curvature of said second portion of said nasal cannula and generate second signals indicative thereof, said controller being in communication with said second sensor, said controller adapted to receive both said first and second signals respectively from said first and second sensors and adjust said actuator between said first and second configurations, said controller adjusting said actuator to said first configuration when at least one of said first and second sensors sense said degree of curvature greater than a threshold value, said controller adjusting said actuator to said second configuration when both said first and second sensors sense said degree of curvature less than said threshold value.

7. The device according to claim 6, wherein said second sensor comprises a resistive flexible sensor having an electrical resistance which changes in response to a change in curvature of said second sensor.

8. The device according to claim 7, wherein said electrical resistance of said second sensor increases with an increase in curvature of said second sensor.

9. The device according to claim 1, wherein said actuator comprises:
a servo motor having a rotatable shaft;
a head mounted on said shaft, said head having at least two fingers extending therefrom, said fingers being in spaced apart relation and defining a slot therebetween, said nasal cannula being received within said slot, said head being rotatable between said first configuration wherein said nasal cannula extend through said slot without deformation thereby permitting flow of said gas therethrough, and said second configuration, wherein said fingers impinge on said nasal cannula so as to form a kink therein thereby preventing flow of said gas therethrough.

10. The device according to claim 1, wherein said controller comprises a microprocessor.

11. The device according to claim 1, further comprising a wireless communication system providing communication between said controller and said first sensor and said actuator.

12. The device according to claim 1, further comprising a first electrical conductor connecting said controller to said first sensor and a second electrical conductor connecting said controller to said actuator.

13. The device according to claim 1, further comprising a power supply electrically connected to said first sensor, said actuator and said controller for providing electrical power thereto.

14. The device according to claim 1, wherein said power supply comprises a battery.

15. The device according to claim 2, wherein said adapter comprises a mask or nasal pillows.

16. (canceled)

17. (canceled)

18. (canceled)

19. (canceled)

20. (canceled)

21. The device according to claim 2, wherein said controller is adapted to generate an alarm signal when said first sensor senses said degree of curvature less than said threshold value.

22. The device according to claim 2, wherein said first sensor comprises a resistive flexible sensor having an electrical resistance which changes in response to a change in curvature of said first sensor.

23. The device according to claim 22, wherein said electrical resistance increases with an increase in curvature of said first sensor.

24. The device according to claim 2, wherein said actuator comprises:
a servo motor having a rotatable shaft;
a head mounted on said shaft, said head having at least two fingers extending therefrom, said fingers being in spaced apart relation and defining a slot therebetween, said tube being received within said slot, said head being rotatable between said first configuration wherein said tube extends through said slot without deformation thereby permitting flow of said gas therethrough, and said second configuration, wherein said fingers impinge on said tube so as to form a kink therein thereby preventing flow of said gas therethrough.
